(19) United States
(12) Patent Application Publication
(10) Pub. No.: US 2023/0051336 A1
(43) Pub. Date: Feb. 16, 2023

(54) QUANTUM DOTS HAVING ACTIVITY OF KILLING MULTIDRUG-RESISTANT BACTERIA (MDR) AND USES THEREOF

(71) Applicant: KOREA ADVANCED INSTITUTE OF SCIENCE AND TECHNOLOGY, Daejeon (KR)

(72) Inventors: Doh Chang LEE, Daejeon (KR); Ilsong LEE, Daejeon (KR); Sungjun KOH, Daejeon (KR); Gui-Min KIM, Daejeon (KR)

(21) Appl. No.: 17/819,473

(22) Filed: Aug. 12, 2022

(30) Foreign Application Priority Data
Aug. 12, 2021 (KR) 10-2021-0106679

(51) Int. Cl. H01L 51/50; B82Y 30/00; B82Y 5/00; H01L 51/00

(52) U.S. Cl. CPC H01L 51/502; B82Y 30/00; B82Y 5/00; H01L 51/0079; H01L 51/0092

(57) ABSTRACT

Disclosed are inorganic nanoparticle quantum dots that effectively kill Gram-positive and Gram-negative bacteria resistant to antibiotics and the treatment of infectious bacterial diseases using the same, and more particularly inorganic nanoparticle quantum dots introduced with a hydrophilic ligand having activity of killing multidrug-resistant bacteria (MDR) and the use thereof. The quantum dots are capable of effectively killing bacteria when used at a low concentration by optimizing the core bandgap thereof and also do not exhibit cytotoxicity, and are thus useful as an agent for preventing or treating infectious diseases caused by multidrug-resistant bacteria.

### CROSS-REFERENCE TO RELATED APPLICATION

The priority under 35 USC § 119 of Korean Patent Application 10-2021-0106679 filed Aug. 12, 2021 is hereby claimed. The disclosure of Korean Patent Application 10-2021-0106679 is hereby incorporated herein by reference, in its entirety, for all purposes.

### TECHNICAL FIELD

The present invention relates to inorganic nanoparticle quantum dots that effectively kill Gram-positive and Gram-negative bacteria resistant to antibiotics and the treatment of infectious bacterial diseases using the same, and more particularly to inorganic nanoparticle quantum dots introduced with a hydrophilic ligand having activity of killing multidrug-resistant bacteria (MDR), a composition for killing multidrug-resistant bacteria or inhibiting the growth thereof comprising the quantum dots, an antibiotic for killing multidrug-resistant bacteria, a disinfectant for killing multidrug-resistant bacteria, a food additive for killing multidrug-resistant bacteria, a composition for preventing or treating an infectious disease caused by multidrug-resistant bacteria, and a method of killing multidrug-resistant bacteria using light.

### BACKGROUND ART

Humans are exposed to the external environment throughout their lifetimes. In particular, microorganisms or viruses are capable of causing fatal diseases in humans. Infectious bacterial diseases are caused by invasion of the human body by pathogenic bacteria, viruses, fungi, parasites, and the like, and treatment thereof is possible using antibiotics and vaccination by virtue of the development of modern medicine. However, the use of antibiotics is becoming increasingly difficult due to the emergence of antibiotic-resistant bacteria.

Antibiotic resistance of bacteria has become a serious problem worldwide, although the extent thereof varies from country to country, and the proportion of resistant bacteria among major bacteria species has increased in recent years. In particular, multidrug resistance of pathogenic bacteria is increasing due to the misuse of antibacterial agents. Specifically, with an increase in the number of multidrug-resistant bacteria (MDR), also called super bacteria, there are many cases in which treatment using antibiotics is difficult, thus becoming a serious health problem. In response thereto, the development of new antibacterial agents is very urgent.

Photodynamic therapy (PDT) is a treatment method that acts in a manner of absorbing light at a specific wavelength using a photosensitizer, generating reactive oxygen species (ROS) through an energy transfer mechanism (Type I), and inactivating or killing surrounding bacteria using the reactive oxygen species.

However, side effects such as damage to normal tissues and the like may occur due to the generated reactive oxygen species after treatment using light, and there are various inconveniences and side effects, such as avoiding exposure to bright sunlight for more than one month after treatment. Moreover, general nanostructured photosensitizers may be decomposed by a stimulus response, thereby limiting the ability thereof to generate reactive oxygen species.

Therefore, a more stable photosensitizer compound and a photodynamic therapy method for inactivating or killing bacteria using the compound having increased ability to generate reactive oxygen species are required.

Against this technical background, the present inventors have made great efforts to develop methods of preventing or treating bacterial infection, which are capable of effectively killing multidrug-resistant bacteria for which there are few antibiotics for treatment when infected with such bacteria that are simultaneously resistant to several types of antibiotics, and also which do not show cytotoxicity, thus solving problems with conventional photodynamic treatment methods, and ascertained that the use of indium phosphide core/zinc selenide shell (InP/ZnSe) or indium phosphide core/zinc sulfide shell (InP/ZnS) quantum dots that are activated under conditions of a specific core bandgap and irradiation with light at a specific wavelength enables multidrug-resistant bacteria to be effectively killed and does not cause cytotoxicity or in-vivo toxicity, thus culminating in the present invention.

The information described in this background section is only for improving understanding of the background of the present invention, and is not to be construed as including information forming the related art already known to those of ordinary skill in the art to which the present invention belongs.

### DISCLOSURE

### Technical Problem

It is an object of the present invention to provide quantum dots for photodynamic therapy (PDT) for the treatment of bacterial infection.

It is another object of the present invention to provide a composition for killing multidrug-resistant bacteria or inhibiting the growth thereof, an antibiotic for killing multidrug-resistant bacteria, a disinfectant for killing multidrug-resistant bacteria, and a food additive for killing multidrug-resistant bacteria, each of which comprises the inorganic nanoparticle quantum dots.

It is still another object of the present invention to provide a composition for preventing or treating an infectious disease caused by multidrug-resistant bacteria comprising the quantum dots, a method of preventing or treating an infectious disease caused by multidrug-resistant bacteria using the quantum dots, the use of the quantum dots for the prevention or treatment of an infectious disease caused by multidrug-resistant bacteria, and the use of the quantum dots for the manufacture of a medicament for the prevention or treatment of an infectious disease caused by multidrug-resistant bacteria.

It is yet another object of the present invention to provide a method of killing multidrug-resistant bacteria comprising the quantum dots using light.

### Technical Solution

In order to accomplish the above objects, the present invention provides inorganic nanoparticle quantum dots introduced with a hydrophilic ligand having activity of killing multidrug-resistant bacteria (MDR).

In addition, the present invention provides a composition for killing multidrug-resistant bacteria or inhibiting the growth thereof comprising the inorganic nanoparticle quantum dots, a method of preventing or treating an infectious disease caused by multidrug-resistant bacteria comprising administering the inorganic nanoparticle quantum dots, the use of the inorganic nanoparticle quantum dots for the prevention or treatment of an infectious disease caused by multidrug-resistant bacteria, and the use of the inorganic nanoparticle quantum dots for the manufacture of a medicament for the prevention or treatment of an infectious disease caused by multidrug-resistant bacteria.

In addition, the present invention provides an antibiotic for killing multidrug-resistant bacteria, a disinfectant for killing multidrug-resistant bacteria, and a food additive for killing multidrug-resistant bacteria, each of which comprises the inorganic nanoparticle quantum dots.

In addition, the present invention provides a method of killing multidrug-resistant bacteria using light comprising (a) mixing the inorganic nanoparticle quantum dots with multidrug-resistant bacteria in vitro and (b) radiating light onto the multidrug-resistant bacteria mixed with the quantum dots.

### DESCRIPTION OF DRAWINGS

FIG. 1 in part A thereof schematically shows a process of preparing InP/ZnS quantum dots and InP/ZnSe quantum dots, which are indium-phosphide-based quantum dots, and in part B thereof shows absorbance spectra of the prepared quantum dots.

FIG. 2 shows the results of analysis of the activity of InP/ZnS quantum dots and InP/ZnSe quantum dots, which are indium-phosphide-based quantum dots, and antibiotics on inhibiting the growth of B. cereus, S. aureus, E. coli, and P. aeruginosa, which are multidrug-resistant bacteria.

FIG. 3 in parts A to E thereof shows the results of analysis of selective reactive oxygen species generation of InP/ZnSe quantum dots, with part A showing results confirming the bactericidal effect on S. aureus after treatment with various reactive oxygen species scavengers, with part B showing the superoxide removal mechanism of TEMPOL, with part C showing the hydroxyl radical removal mechanism of IPA, with part D showing the singlet oxygen removal mechanism of azide, and with part E showing the hydrogen peroxide removal mechanism of ferric EDTA.

FIG. 4 in parts A to C thereof shows the results of evaluation of animal cytotoxicity of InP/ZnS and InP/ZnSe quantum dots, which are indium phosphide quantum dots, with part A showing toxicity to monkey-kidney-derived fibroblasts, with part B showing toxicity to human-derived dermal fibroblasts, and with part C showing toxicity to human-derived skin epidermal cells.

FIG. 5 in parts A and B shows the results of pharmacokinetic analysis of InP/ZnS quantum dots and InP/ZnSe quantum dots, which are indium-phosphide-based quantum dots, with part A showing InP/ZnS quantum dots, and with part B showing InP/ZnSe quantum dots.

FIG. 6 in parts A and B shows the results of analysis of bactericidal kinetics for S. aureus of InP/ZnS quantum dots and InP/ZnSe quantum dots, which are indium-phosphide-based quantum dots, with part A showing the results of analysis of bactericidal kinetics of InP/ZnS quantum dots and InP/ZnSe quantum dots, and with part B showing the bactericidal effect of InP/ZnS quantum dots and InP/ZnSe quantum dots represented by the colony-forming units.

FIG. 7 in parts A to C shows the effect of InP/ZnSe quantum dots on treating S. aureus infection in a mouse model, with part A showing changes in the size of a wound in the infected group, the group irradiated only with light after infection, the group that caused only the wound, the group subjected to PDT for 15 minutes after infection, and the group subjected to PDT for 60 minutes after infection, and results confirming bacteria present in the wound tissue through a bacterial culture test after 6 days, with part B being a graph showing changes in the size of the wound in the experimental groups, and with part C being a graph showing the results of the bacterial culture test.

FIG. 8 in parts A to E shows results comparing the difference in bactericidal activity depending on the core bandgap and the concentration of quantum dots, with part A showing absorbance spectra of InP/ZnS quantum dots and InP/ZnSe quantum dots having the adjusted InP core bandgap, with part B schematically showing changes in the size of the InP core having the adjusted bandgap, with part C showing the bactericidal effect of InP/ZnSe quantum dots having the adjusted InP core bandgap on MDR E. coli and electron/hole wave function overlap integrals, with part D showing cytotoxicity of the quantum dots in HaCaT cells, and with part E showing the results of analysis of hemolysis by InP/ZnSe quantum dots.

FIG. 9 in parts A and B shows results comparing the bactericidal activity depending on the bandgap of quantum dots, with part A showing absorbance spectra and bandgap of InP/ZnSe quantum dots having the adjusted InP core band gap, and with part B showing the bactericidal effect of InP/ZnSe quantum dots having the adjusted InP core band gap on MDR S. aureus.

### DETAILED DESCRIPTION AND EXEMPLARY EMBODIMENTS

Unless otherwise defined, all technical and scientific terms used herein have the same meanings as those typically understood by those skilled in the art to which the present invention belongs. In general, the nomenclature used herein is well known in the art and is typical.

In an embodiment of the present invention, it has been confirmed that, even when InP/ZnSe quantum dots or InP/ZnS quantum dots introduced with a hydrophilic ligand MPA are used at a low concentration, B. cereus, S. aureus, E. coli, and P. aeruginosa, which are multidrug-resistant bacteria, may be effectively killed and the growth thereof may be inhibited, and also that only multidrug-resistant bacteria are selectively killed without cytotoxicity through animal model experiments.

Accordingly, an aspect of the present invention pertains to inorganic nanoparticle quantum dots introduced with a hydrophilic ligand having activity of killing multidrug-resistant bacteria (MDR).

In the present invention, the inorganic nanoparticle quantum dots may be quantum dots having an indium phosphide core/zinc selenide shell (InP/ZnSe) or an indium phosphide core/zinc sulfide shell (InP/ZnS), and preferably quantum dots having an indium phosphide core/zinc sulfide shell (InP/ZnS), but the present invention is not limited thereto (see FIG. 1 in parts A and B thereof).

In the present invention, the inorganic nanoparticle quantum dots may have a core bandgap of 2 eV to 3 eV, preferably 2.03 eV to 2.71 eV, more preferably 2.46 eV to 2.71 eV, and most preferably 2.71 eV, but the present invention is not limited thereto.

Due to a difference in the core bandgap, electron/hole wave function overlap integrals vary inside the quantum dots (FIG. 8 in part C, red scatter-line graph). When the overlap integrals increase, a type I structure in which electrons and holes in the quantum dots are confined to the core may be formed, whereas when the overlap decreases, a quasi-type II structure in which electrons are delocalized to the shell to thus facilitate electron extraction may be formed. The bactericidal effect may be enhanced only when a superoxide is effectively formed by extracting the electrons generated inside the quantum dots and reducing oxygen, thereby confirming that, as the core size increases and the bandgap decreases, overlap integrals increase and bacterial survival increases.

According to an embodiment of the present invention, the survival rate in the bactericidal experiment using InP/ZnSe having the largest bandgap was about 70% within 1 hour, whereas the survival rate was decreased to 1% or less within 1 hour in the experiment using InP/ZnSe having the smallest core bandgap. Consequently, it was found that the most effective bactericidal effect was exhibited when the core bandgap of the quantum dots was 2.71 eV (the rightmost bar in the bar graph of part C of FIG. 8).

In the present invention, reactive oxygen species (ROS) generated by irradiating the inorganic nanoparticle quantum dots with light at a wavelength of 300 nm to 500 nm is capable of killing multidrug-resistant bacteria or inhibiting the growth thereof.

The wavelength of the irradiated light is preferably 350 nm to 450 nm, more preferably 400 nm, but is not limited thereto.

In the present invention, the reactive oxygen species (ROS) may be singlet oxygen, superoxide, perhydroxyl radical, hydroxyl radical, or mixtures thereof.

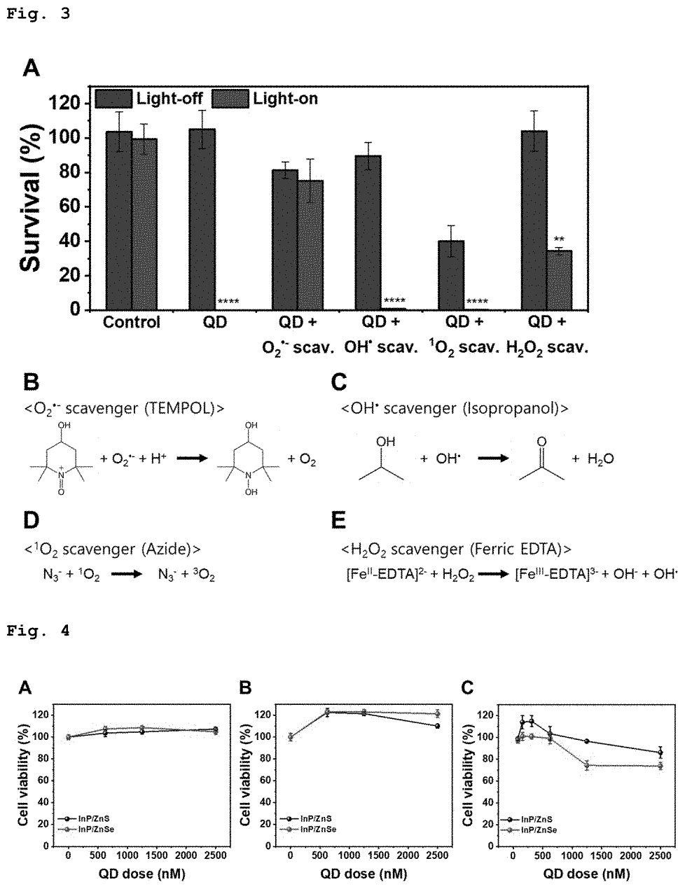

In an embodiment of the present invention, it has been confirmed that, when light at a wavelength of 400 nm was radiated onto the antibiotic-resistant bacteria group treated with quantum dots including the indium phosphide core, a superoxide was generated, so bacterial death occurred through direct interaction between quantum dots and bacteria (see FIG. 3 in parts A to E thereof).

In the present invention, the hydrophilic ligand may be selected from the group consisting of 3-mercaptopropionic acid (MPA), L-glutathione (GSH), mercaptoacetic acid, mercaptobutanoic acid, mercaptopentanoic acid, mercaptohexanoic acid, mercaptoheptanoic acid, mercaptooctanoic acid, mercaptononanoic acid, mercaptodecanoic acid, mercaptoundecanoic acid, mercaptododecanoic acid, and L-cysteine, and is preferably 3-mercaptopropionic acid, but the present invention is not limited thereto.

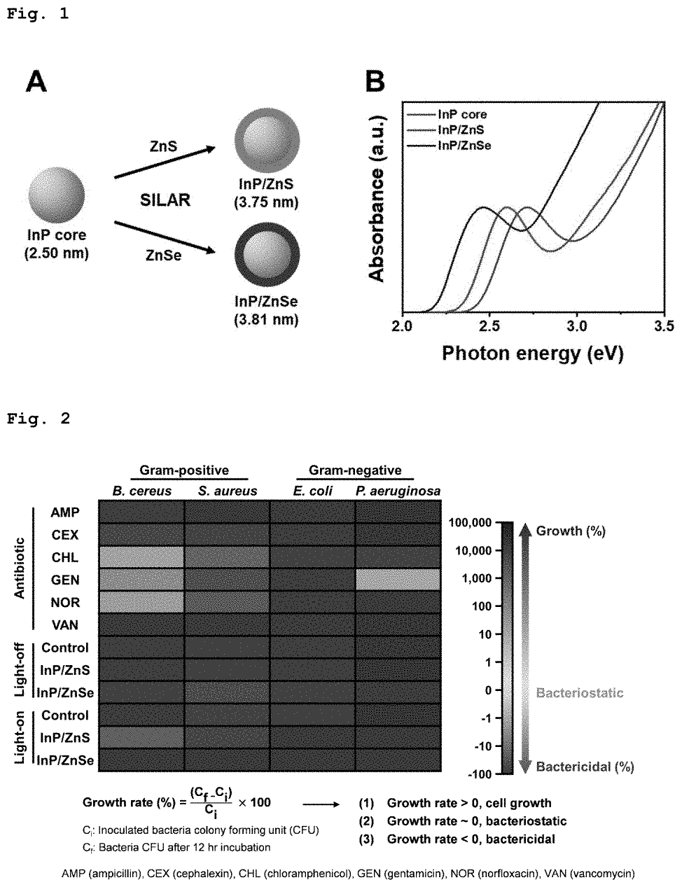

In the present invention, the multidrug-resistant bacteria may be selected from the group consisting of Bacillus cereus (B. cereus), Staphylococcus aureus (S. aureus), Escherichia coli (E. coli), Pseudomonas aeruginosa (P. aeruginosa), Acinetobacter baumannii, Klebsiella pneumonia, Enterococcus faecium, Enterobacteriaceae, Helicobacter pylori, Campylobacter spp., Salmonellae, Neisseria gonorrhoeae, Streptococcus pneumoniae, Haemophilus influenzae, and Shigella spp., and are preferably B. cereus, S. aureus, E. coli, or P. aeruginosa, but the present invention is not limited thereto (FIG. 2).

In the present invention, the term “multidrug-resistant bacteria (MDR)” is interchangeable with “super bacteria”, and refers to bacteria that are simultaneously resistant to several types of antibiotics.

In the present invention, the multidrug-resistant bacteria may include Gram-positive bacteria and Gram-negative bacteria, and may also be resistant to antibiotics based on penicillin, cephalosporins, amphenicols, aminoglycosides, quinolones, and/or glycopeptides, particularly to ampicillin (AMP), cephalexin (CEX), chloramphenicol (CHL), gentamicin (GEN), norfloxacin (NOR), and/or vancomycin (VAN), but the present invention is not limited thereto.

Another aspect of the present invention pertains to a composition for killing multidrug-resistant bacteria or inhibiting the growth thereof comprising the inorganic nanoparticle quantum dots.

In an embodiment of the present invention, it has been confirmed that, when multidrug-resistant bacteria were cultured in an LB medium treated with quantum dots and were then irradiated with light, survival of multidrug-resistant bacteria was greatly reduced.

Still another aspect of the present invention pertains to an antibiotic for killing multidrug-resistant bacteria comprising the inorganic nanoparticle quantum dots.

As used herein, the term “antibiotic” includes a preservative, bactericide, and antibacterial agent for pharmaceutical or cosmetic use, and may be useful as an antibiotic having an excellent antibacterial effect, bactericidal effect, or antiseptic effect.

Yet another aspect of the present invention pertains to a disinfectant for killing multidrug-resistant bacteria comprising the inorganic nanoparticle quantum dots. The disinfectant may be useful as a disinfectant for health and hospitals to prevent disease transmission in hospitals, and may also be used as a disinfectant for general life, a disinfectant for food, kitchens, and food service equipment, and a disinfectant for livestock in the livestock industry.

Still yet another aspect of the present invention pertains to a food additive for killing multidrug-resistant bacteria comprising the inorganic nanoparticle quantum dots.

Examples of the food additive of the present invention may include preservatives, sanitizers, antioxidants, spices, seasonings, sweeteners, fragrances, expanding agents, fortifying agents, improving agents, emulsifiers, various nutrients, flavoring agents such as synthetic flavoring agents and natural flavoring agents, colorants, color-developing agents, thickeners (cheese, chocolate, etc.), pectic acid and salts thereof, alginic acid and salts thereof, organic acids, protective colloidal thickeners, pH adjusters, stabilizers, defoamers, solvents, release agents, antiseptic agents, quality improvers, glycerin, alcohols, and carbonation agents for use in carbonated beverages, which may be additionally added to food through immersion, spraying, or mixing.

A further aspect of the present invention pertains to a composition for preventing or treating an infectious disease caused by multidrug-resistant bacteria comprising the inorganic nanoparticle quantum dots.

Still a further aspect of the present invention pertains to a method of preventing or treating an infectious disease caused by multidrug-resistant bacteria comprising administering the inorganic nanoparticle quantum dots.

Yet a further aspect of the present invention pertains to the use of the inorganic nanoparticle quantum dots for the prevention or treatment of an infectious disease caused by multidrug-resistant bacteria.

Still yet a further aspect of the present invention pertains to the use of the inorganic nanoparticle quantum dots for the manufacture of a medicament for the prevention or treatment of an infectious disease caused by multidrug-resistant bacteria.

In the present invention, the concentration of the inorganic nanoparticle quantum dots may be 50 nM to 200 nM, preferably 50 to 150 nM, more preferably 100 nM, but is not limited thereto.

In an embodiment of the present invention, the concentration of the quantum dots in an animal infection experimental model was set to 100 nM. There are prior documents that report testing the quantum dots at a concentration of 1-4 μM (Colleen R. McCollum, et al., ACS Appl. Mater. Interfaces 2021), but based on the experimental results of the present invention, animal cell viability was decreased to 95% or less at a quantum dot concentration of 625 nM or more (FIG. 8 in part D thereof), and hemolysis of red blood cells was shown by the quantum dots at a concentration of 1000 nM or more. Consequently, the inorganic nanoparticle quantum dots according to the present invention were found to exhibit remarkable bactericidal activity while causing hardly any cytotoxicity, even when used at very low concentrations (FIGS. 4 to 7).

In the present invention, the infectious disease caused by multidrug-resistant bacteria may be pneumonia, sepsis, urinary tract infection, food poisoning, impetigo, purulent disease, acute dermatitis, wound infection, bacteremia, endocarditis, or enteritis, but is not limited thereto.

As used here, the term “prevention” refers to any action that inhibits or delays an infectious disease caused by multidrug-resistant bacteria upon administration of the composition comprising the quantum dots. In addition, the term “treatment” used herein refers to any action in which the symptoms of an infectious disease caused by multidrug-resistant bacteria are ameliorated or eliminated upon administration of the composition comprising the quantum dots.

The composition for preventing or treating an infectious disease caused by multidrug-resistant bacteria according to the present invention may comprise a pharmaceutically effective amount of the quantum dots alone, or may also comprise one or more pharmaceutically acceptable carriers, excipients, or diluents. Here, the pharmaceutically effective amount is an amount sufficient to prevent, ameliorate, or treat the symptoms of an infectious disease caused by multidrug-resistant bacteria.

The term “pharmaceutically acceptable” refers to a composition that is physiologically acceptable and does not normally cause allergic reactions such as gastrointestinal disorders and dizziness or similar reactions when administered to humans. Examples of such carriers, excipients, and diluents may include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methylhydroxybenzoate, propylhydroxybenzoate, talc, magnesium stearate, and mineral oil. Also, fillers, anticoagulants, lubricants, wetting agents, fragrances, emulsifiers, and preservatives may be further included.

The term “carrier” refers to a material that facilitates addition of a compound into a cell or tissue. The term “diluent” is defined as a material that stabilizes the biologically active form of the compound of interest and is diluted with the water in which the compound is dissolved.

The composition of the present invention may be formulated using methods known in the art to provide rapid, sustained, or delayed release of the active ingredient after administration to an animal. Formulations may be in the form of powders, granules, tablets, emulsions, syrups, aerosols, soft or hard gelatin capsules, sterile injectable solutions, or sterile powders.

The composition of the present invention may be administered via various routes, including oral, transdermal, subcutaneous, intravenous, or intramuscular routes, and the dosage of the active ingredient may be appropriately determined depending on various factors, such as the route of administration, the patient's age, gender, and body weight, and the severity of disease. The composition according to the present invention may be administered in combination with a known compound having an effect of preventing, ameliorating, or treating the symptoms of an infectious disease caused by multidrug-resistant bacteria.

Even a further aspect of the present invention pertains to a method of killing multidrug-resistant bacteria using light comprising (a) mixing the inorganic nanoparticle quantum dots with multidrug-resistant bacteria in vitro and (b) radiating light onto the multidrug-resistant bacteria mixed with the quantum dots.

The concentration of the inorganic nanoparticle quantum dots in step (a) may be 50 nM to 200 nM, preferably 50 to 150 nM, more preferably 100 nM, but is not limited thereto.

In the present invention, in step (b), light may be applied at a wavelength of 300 nm to 500 nm. Although not limited thereto, the wavelength is preferably 350 nm to 450 nm, more preferably 400 nm, and reactive oxygen species (ROS) generated through light irradiation are able to kill multidrug-resistant bacteria or inhibit the growth thereof.

Hereinafter, the present invention will be described in more detail with reference to the following examples. However, it will be obvious to those skilled in the art that the following examples are provided only for illustration of the present invention and should not be construed as limiting the scope of the present invention.

### Example 1: Preparation of Indium-Phosphide-Based Quantum Dots

The process of preparing indium-phosphide-based quantum dots having activity of killing antibiotic-resistant bacteria according to the present invention and the absorbance spectra thereof are shown in FIG. 1 in parts A and B thereof, respectively.

### Example 1-1: Preparation of Indium Phosphide Quantum Dot Core

In order to prepare an indium phosphide (InP) quantum dot core, 17 mL of 1-octadecene (ODE) was placed in a round-bottom three-necked flask, after which 292 mg (1 mmol) of an indium acetate powder, 1264.7 mg (2 mmol) of a zinc stearate powder, and 853.4 mg (3 mmol) of a stearic acid powder were added thereto, the temperature was raised to 120° C., and degassing was performed. Thereafter, the inside of the flask was purged with argon (Ar) to create an argon (Ar) atmosphere, and 2 mL of trioctylphosphine (TOP) was added thereto, following by cooling to room temperature. 1 mL of a phosphine precursor solution, obtained by dissolving tris(trimethylsilyl)phosphine ((TMP)₃P) at a concentration of 0.5 M in ODE, was added at room temperature, the temperature was raised to 300° C., and reaction was carried out for 10 minutes.

### Examples 1-2: Preparation of Indium Phosphide Core/Zinc Sulfide Shell (InP/ZnS) Quantum Dots

19 mL of the InP core quantum dot solution prepared in Example 1-1 was placed in a round-bottom three-necked flask, the temperature was raised to 120° C., and degassing was performed. After degassing, the temperature was raised to 300° C., and 0.96 mL of each of a zinc precursor and a sulfur precursor was added thereto, followed by reaction for 30 minutes. Thereafter, 1.4 mL of each of the zinc precursor and the sulfur precursor was further added thereto, followed by reaction for 30 minutes. The zinc precursor used in the reaction was 0.5 M zinc oleate including 1 M TOP, and the sulfur precursor was a solution (TOP(S)) in which 0.5 M sulfur was dissolved in TOP.

### Examples 1-3: Preparation of Indium Phosphide Core/Zinc Selenide Shell (InP/ZnSe) Quantum Dots

19 mL of the InP core quantum dot solution prepared in Example 1-1 was placed in a round-bottom three-necked flask, the temperature was raised to 120° C., and degassing was performed. After degassing, the temperature was raised to 300° C., and 0.89 mL of each of a zinc precursor and a selenium precursor was added thereto, followed by reaction for 30 minutes. Thereafter, 1.4 mL of each of the zinc precursor and the selenium precursor was further added thereto, followed by reaction for 30 minutes. The zinc precursor used in the reaction was 0.5 M zinc oleate including 1 M TOP, and the selenium precursor was a solution (TOP(Se)) in which 0.5 M selenium was dissolved in TOP.

### Examples 1-4: Hydrophilic Ligand Substitution in Indium-Phosphide-Based Quantum Dots

Since the surfaces of the InP/ZnS quantum dots and the InP/ZnSe quantum dots prepared in Examples 1-2 and 1-3 were passivated with a hydrophobic ligand, they are not suitable for use in killing antibiotic-resistant bacteria, and thus substitution with a hydrophilic ligand has to be performed. The hydrophilic ligand precursor solution was a solution of 0.5 M 3-mercaptopropionic acid (MPA) dissolved in methanol, and was added with tetramethylammonium hydroxide (TMAH) in order to adjust the pH thereof to 10-12.

1 mL of the InP/ZnS quantum dot solution prepared in Example 1-2 or the InP/ZnSe quantum dot solution prepared in Example 1-3 was added to 5 mL of the hydrophilic ligand precursor solution, followed by sonication in a sonicator for 10 minutes. After ultrasonic dispersion, the quantum dot solution having the substituted surface ligand was treated with methanol, hexane, or acetone and then centrifuged at 10,000 rpm for 5 minutes to precipitate quantum dots. The quantum dot precipitation process using methanol, hexane, or acetone was repeated at least 3 times to remove impurities.

### Example 2: Evaluation of Efficacy of Indium-Phosphide-Based Quantum Dots on Inhibiting Growth of Antibiotic-Resistant Bacteria

In order to compare the efficacy of the InP/ZnS quantum dots and the InP/ZnSe quantum dots prepared in Example 1 on inhibiting the growth of antibiotic-resistant bacteria and the performance thereof with antibiotics, a bacterial culture test was performed in a quantum-dot- or antibiotic-treated LB (Luria-Bertani broth) medium for 10 hours, and the results thereof are shown in FIG. 2. The initial concentrations of the inoculated bacteria in vitro in FIG. 2 were 3.5×10⁶ CFU/mL of Bacillus cereus (B. cereus), 1.4×10⁷ CFU/mL of Staphylococcus aureus (S. aureus), 5.3×10⁶ CFU/mL of Escherichia coli (E. coli), and 2.6×10⁷ CFU/mL of Pseudomonas aeruginosa (P. aeruginosa), and the concentrations of quantum dots and antibiotics administered in vitro were 0.39 μg/mL (10 nM) of InP/ZnS, 0.50 μg/mL (10 nM) of InP/ZnSe, 12.5 μg/mL of ampicillin (AMP), 6.25 μg/mL of cephalexin (CEX), 25 μg/mL of chloramphenicol (CHL), 6.25 μg/mL of gentamicin (GEN), 6.25 μg/mL of norfloxacin (NOR), and 3.125 μg/mL of vancomycin (VAN), all of which were higher than the antibiotic sensitivity concentrations set forth in 2020 CLSI (Clinical and Laboratory Standard Institute) M100 (30ₜₕ ed.). All the bacteria used in the test were purchased from an antibiotic-resistant strain bank (B. cereus CCARM 0002, S. aureus CCARM 0205, E. coli CCARM 1B700, and P. aeruginosa CCARM 0219).

The administered AMP was penicillin, CEX was cephalosporins, CHL was amphenicols, GEN was aminoglycosides, NOR was quinolones, and VAN was glycopeptides, all of which were different types of antibiotics. All of B. cereus, S. aureus, E. coli, and P. aeruginosa were multidrug-resistant bacteria (MDR) resistant to three or more types of antibiotics, and bacteriostatic and bactericidal effects of most antibiotics thereon were not observed. Bacterial growth occurred both in the group not treated with quantum dots and in the group treated with quantum dots, whereas the group irradiated with light at a wavelength of 400 nm after treatment with InP/ZnS quantum dots exhibited bactericidal effects on E. coli and P. aeruginosa, and the group irradiated with light at a wavelength of 400 nm after treatment with InP/ZnSe quantum dots exhibited bactericidal effects on all four types of bacteria, confirming that they are effective at treating multidrug-resistant bacterial infections for which antibiotics are no longer effective.

### Example 3: Evaluation of Ability of Indium-Phosphide-Based Quantum Dots to Selectively Generate Reactive Oxygen Species

In order to evaluate the ability of the InP/ZnSe quantum dots prepared in Example 1 to selectively generate reactive oxygen species (ROS), bacteria were cultured for 1 hour in PBS treated with various types of ROS scavengers. The results thereof are shown in FIG. 3 in part A thereof. The bacteria used in FIG. 3 in parts A to E thereof were S. aureus, and the final concentration of the inoculated bacteria in vitro was 4.1×10⁷ CFU/mL. The mechanisms of action of ROS scavengers are shown in FIG. 3 in parts B to E thereof, and ROS scavenger concentrations were 2 mM 4-hydroxy-2,2,6,6-tetramethylpiperidine-1-oxyl (TEMPOL), 0.5 mM isopropanol (IPA), 320 mM sodium azide (NaN₃), and 9.6 mM ferric EDTA.

InP quantum dots cannot form a hydroxyl radical (OH·), which is highly toxic to animal cells in the valance band (VB) due to the intrinsic energy level thereof but are easily used to form a superoxide (O₂^{·-}) by reducing oxygen in the conduction band (CB). A superoxide not only acts as a signal transmitter such as a peptide growth factor and differentiation factor in vivo, but is also made by NADPH oxidase and used to defend infection against bacteria. The superoxide thus generated is cleared by superoxide dismutase (SOD) and catalase (CAT), and does not harm animal cells.

The experimental group not treated with the scavenger showed a decrease in bacterial survival to 0.01% (p<0.005) when irradiated with light, whereas the experimental group treated with TEMPOL did not show a great difference in survival between the group irradiated with light and the group not irradiated with light (p=0.563). All the groups treated with IPA, NaN₃, and ferric EDTA showed a significant decrease in survival (IPA: p<0.005, NaN₃: p<0.005, ferric EDTA: p<0.05). Thereby, it was confirmed that the superoxide is selectively generated by the quantum dots and is thus capable of effectively killing bacteria.

### Example 4: Evaluation of Cytotoxicity of Indium-Phosphide-Based Quantum Dots

Inorganic nanoparticles are known to exhibit cytotoxicity. Although it is known that indium-phosphide-based quantum dots are less toxic to the human body and the environment compared to conventional cadmium-based quantum dots, quantum dots must not be cytotoxic in order to be usable as a therapeutic agent for infection in vivo. Accordingly, the cytotoxicity of the InP/ZnS and InP/ZnSe quantum dots of Example 1 according to the present invention was evaluated, and the results thereof are shown in FIG. 4 in parts A to C thereof.

FIG. 4 in part A shows the results of evaluation of cytotoxicity to monkey-kidney-tissue-derived fibroblasts (COS-7), FIG. 4 in part B shows the results of evaluation of cytotoxicity to human-derived dermal fibroblasts (HDF), and FIG. 4 in part C shows the results of evaluation of cytotoxicity to human-derived skin epidermal cells (HaCaT). As shown in FIG. 4 in parts A to C thereof, both the InP/ZnS quantum dots and the InP/ZnSe quantum dots according to Example 1 showed cell viability of 95% or more in COS-7 and HDF and cell viability of 95% or more in HaCaT at a quantum dot concentration of 625 nM or less. Based thereon, it was confirmed that the quantum dots of Example 1 were not cytotoxic.

### Example 5: Pharmacokinetic Analysis of Indium-Phosphide-Based Quantum Dots

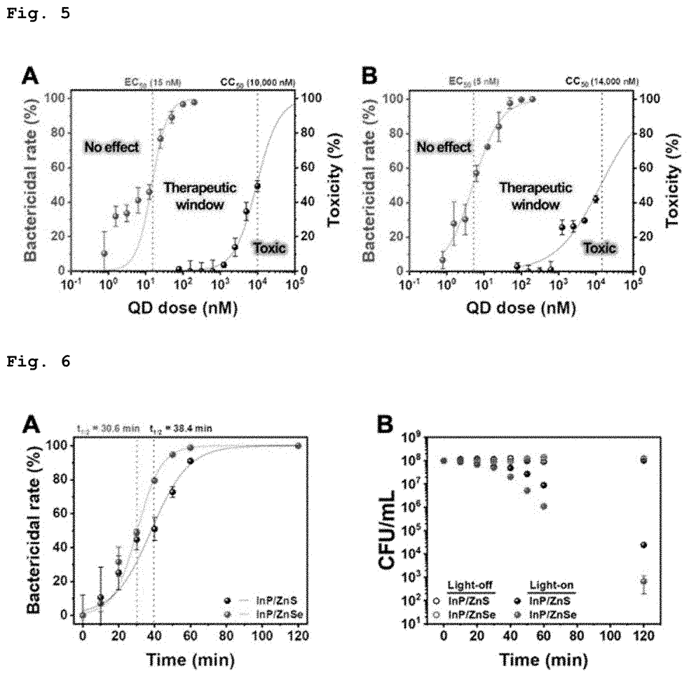

As the concentration of a drug that is administered is increased, the expected effect increases, but the drug may cause toxicity in vivo above a predetermined concentration. Hence, in order to confirm the in-vivo application concentration in the treatment of infection using quantum dots, in-vitro pharmacokinetic analysis of the quantum dots prepared in Example 1 was performed, and the results thereof are shown in FIG. 5 in parts A and B thereof. Bacteria and animal cells used for the analysis were S. aureus of Example 2 and HaCaT of Example 4, respectively, and the bactericidal effect depending on the concentration was obtained by counting colony-forming units after treatment with quantum dots and then irradiation with light for 1 hour, and the initial concentration of S. aureus was 9.9×10⁷ CFU/mL.

FIG. 5 in part A shows the results of pharmacokinetic analysis for the InP/ZnS quantum dots of Example 1, and FIG. 5 in part B shows the results of pharmacokinetic analysis for the InP/ZnSe quantum dots of Example 1. As shown in FIG. 5 in parts A and B thereof, the half maximal effective concentration (EC₅₀) and the half maximal cytotoxicity concentration (CC₅₀) by the InP/ZnS quantum dots were 15 nM (0.59 μg/mL) and 10,000 nM (390 μg/mL), respectively, and the selectivity index (SI=CC₅₀/EC₅₀) was 667. EC₅₀ and CC₅₀ by the InP/ZnSe quantum dots were 5 nM (0.25 μg/mL) and 14,000 nM (1,400 μg/mL), respectively, and the selectivity index was 2,800. Based thereon, it can be confirmed that InP/ZnSe exhibited a wider therapeutic window than InP/ZnS, and the bactericidal effect of 50% was manifested within 1 hour when using InP/ZnS at 15 nM (0.59 μg/mL) and InP/ZnSe at 5 nM (0.25 μg/mL) or more. The bactericidal effect at various concentrations and the cytotoxic concentration range were shown based on the pharmacokinetic analysis results.

### Example 6: Bactericidal Kinetic Analysis of Indium-Phosphide-Based Quantum Dots

In the treatment of PDT-based bacterial infection using quantum dots, it is necessary to clearly analyze the quantum dot treatment time and the light irradiation time to set the effective therapeutic time. To this end, the bactericidal effect depending on the time was analyzed through bactericidal kinetics of the InP/ZnS quantum dots and the InP/ZnSe quantum dots of Example 1, and the results thereof are shown in FIG. 6 in parts A and B thereof. The bacteria used for the analysis were S. aureus of Example 2, and the initial concentration thereof was 9.9×10⁷ CFU/mL. When the bactericidal kinetic analysis was performed by setting the bactericidal concentration of the quantum dots to 100 nM through the pharmacokinetic analysis of Example 5, the half-life (t_{1/2}) of the InP/ZnS quantum dots was 38.4 minutes, and the bactericidal effect thereof was 91% within 1 hour and 99.97% within 2 hours. The half-life of the InP/ZnSe quantum dots was 30.6 minutes, and the bactericidal effect thereof was 99% within 1 hour and 99.999% within 2 hours.

### Example 7: Analysis of In-Vivo Bacterial Infection Therapeutic Effect of Indium-Phosphide-Based Quantum Dots

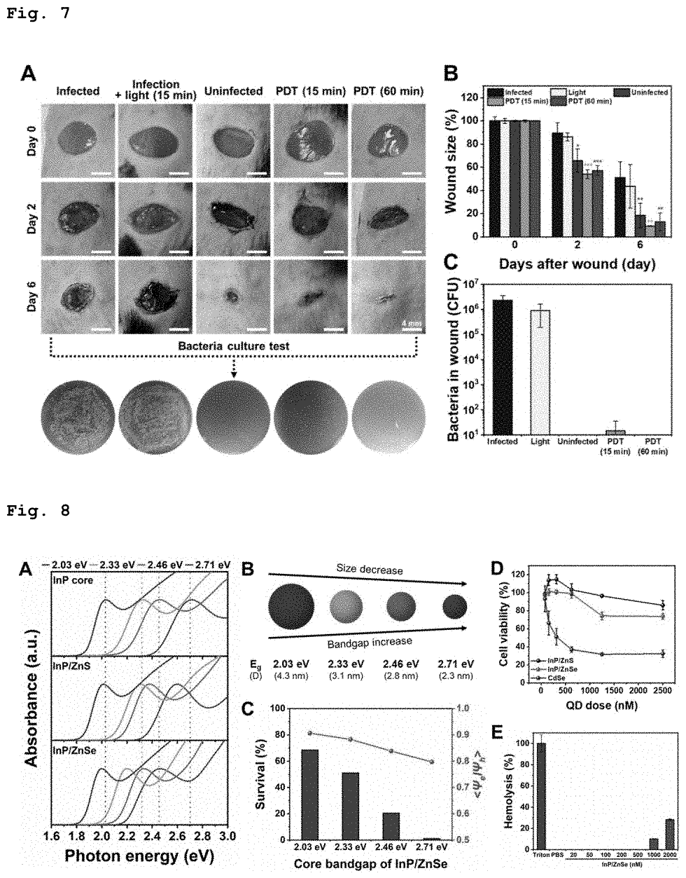

In order to verify the in-vivo effect of the PDT-based bacterial infection treatment method using quantum dots, the concentration of administered InP/ZnSe quantum dots was set to 100 nM based on the results of Example 5, the treatment times were set to 15 minutes and 60 minutes based on the results of Example 6, and an infection treatment experiment was performed in a mouse infection model. The results thereof are shown in FIG. 7 in parts A to C thereof.

In mouse infection models, wound recovery was slow in the group without additional treatment after infection and in the group irradiated with light after infection. In contrast, when PDT was performed for 15 or 60 minutes after treatment with InP/ZnSe quantum dots, the size of the wound was decreased after 6 days to a level similar to that of the uninfected model. The results thereof are shown in FIG. 7 in parts A and B thereof.

In order to quantitatively measure the bacteria in the infected wound tissue, a section of the wound tissue was collected and a bacterial culture test was performed, and the results thereof are shown in FIG. 7 in part C thereof. Like the trend of reduction of the wound size shown in FIG. 7 in parts A and B thereof, bacteria were detected both in the infected group with slow wound recovery and in the group only irradiated with light after infection, whereas no bacteria were detected in the uninfected group or in the group subjected to PDT for 60 minutes, and also a small amount of bacteria was detected in the group subjected to PDT for 15 minutes. Thereby, it was confirmed that a superoxide could be selectively generated in vivo through PDT using quantum dots to thus exhibit bactericidal effects and be effective for treatment and recovery of infected wounds.

### Example 8: Evaluation of Bactericidal Effect and Cytotoxicity of Indium-Phosphide-Based Quantum Dots with Optimized Energy Level and Treatment Concentration

The bandgap of quantum dots can be controlled by adjusting the size thereof, which causes the electron/hole distribution in the quantum dots to change, and thus electron/hole overlap integrals vary. When the electron/hole overlap integrals increase due to a change in the electron/hole distribution of the quantum dots, a type I structure in which the electrons and holes in the quantum dots are confined to the core is formed. In contrast, when the overlap integrals decrease, a quasi-type II structure in which electrons are delocalized in the entire quantum dot region and holes are confined to the core is formed, making it easy to extract electrons therefrom. Hence, the distribution of electrons/holes is important in the reaction for extracting and using electrons and holes from quantum dots, which greatly affects the bactericidal effect of quantum dots. In order to analyze the energy level of quantum dots optimized for bactericidal effects, the bactericidal effect of InP/ZnSe quantum dots on MDR E. coli was analyzed, and the results thereof are shown in FIG. 8 in parts A to E thereof.

FIG. 8 in part A shows the absorbance spectra of the quantum dots having the adjusted core bandgap, and FIG. 8 in part B shows the bandgap and size of the core based thereon. The diameters of the InP/ZnSe quantum dots configured such that the ZnSe shell was grown on quantum dots having core bandgaps of 2.03, 2.33, 2.46, and 2.71 eV are 5.6, 4.4, 4.1, and 3.6 nm, respectively.

FIG. 8 in part C shows the electron/hole overlap integrals of InP/ZnSe quantum dots having the adjusted core bandgap and the bactericidal effect thereof on MDR E. coli. Four types of InP/ZnSe quantum dots, in which the absorbance of light at a wavelength of 400 nm was fixed to 0.025 and thus the amount of absorbed light was made equal to the number of generated excitons, were used to treat MDR E. coil at a concentration of 2×10⁸ CFU/mL, followed by irradiation with light at 400 nm for 1 hour. Therefore, the quantum dots having the largest core bandgap exhibited the greatest bactericidal effect. The electron/hole overlap integrals of the quantum dots were the smallest in the quantum dots in which the difference in conduction band energy level between the InP core and the ZnSe shell was the smallest due to the largest core bandgap, indicating the smallest electron/hole overlap. Based thereon, it was confirmed that, when the core bandgap of the quantum dots increased and thus the electron/hole overlap became smaller, electron extraction was easy, and bacteria could be effectively killed.

It is necessary to more precisely determine the toxic concentration and hemolytic concentration of the quantum dots in order to treat infection with the quantum dots at a concentration confirmed to be safe in vivo. To this end, cytotoxicity and hemolysis of InP/ZnSe quantum dots were analyzed, and the results thereof are shown in FIG. 8 in parts D and E thereof. FIG. 8 in part D shows the cytotoxicity of InP/ZnSe quantum dots in HaCaT cells, and FIG. 8 in part E shows the results of analysis of hemolysis of red blood cells by InP/ZnSe quantum dots. Based on the results of analysis of cytotoxicity of FIG. 8 in part D thereof, it was confirmed that the cell viability was decreased to 95% or less in the presence of InP/ZnSe quantum dots at 625 nM or higher, and also that hemolysis of red blood cells appeared in the presence of InP/ZnSe quantum dots at a concentration of 1,000 nM or higher. Based thereon, it was found that the use of InP/ZnSe quantum dots at a concentration of 625 nM or higher for the treatment of infection in vivo is capable of causing in-vivo toxicity.

### Example 9: Comparison of Bactericidal Effect of Indium-Phosphide-Based Quantum Dots with Controlled Energy Level

Bactericidal effects according to the difference in band gap were compared. InP/ZnSe quantum dots with a band gap of 1.88 eV were additionally produced, and the quantum dots are materials in which the band gap is red shifted to 1.88 eV after ZnSe shell passivation on an InP core with a core band gap of 1.9 eV. The InP/ZnSe quantum dots having a band gap of 2.46 eV according to the present invention are obtained by passivation of a ZnSe shell to an InP core having a band gap of 2.71 eV.

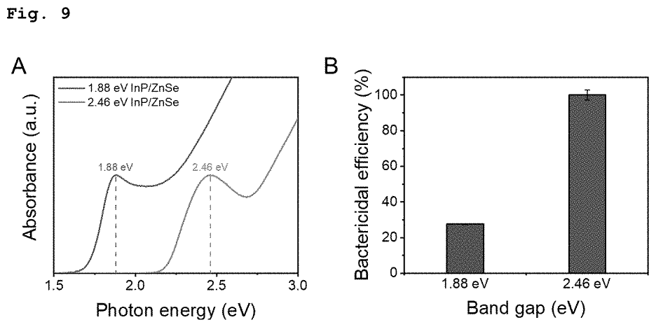

FIG. 9 in part A thereof is an absorbance spectra and band gap energy of InP/ZnSe quantum dots in which a ZnSe shell is grown on indium phosphide quantum dots having a core band gap of 2.71 eV or 1.90 eV, respectively. The band gap energies of InP/ZnSe quantum dots prepared using indium phosphide quantum dots with core band gaps of 2.71 eV and 1.90 eV are 2.46 eV and 1.88 eV, respectively.

FIG. 9 in part B thereof shows the bactericidal activity of MDR S. aureus bacteria by quantum dots having band gap energies of 2.46 eV and 1.88 eV, respectively. When two InP/ZnSe quantum dots with the same concentration of 100 nM were treated with MDR S. aureus at a concentration of 2×10⁸ CFU/mL and then irradiated with 400 nm light for one hour, InP/ZnSe quantum dots with a band gap of 2.46 eV showed the bactericidal effect of 99.999%, and Inp/ZnSe quantum dots with a band gap of 1.88 eV showed the bactericidal effect of 27.58%.

Therefore, it can be seen that the bactericidal effect of quantum dots with a band gap of 2.46 eV is significantly increased compared to those with a band gap of 1.88 eV.

### INDUSTRIAL AVAILABILITY

As is apparent from the above description, the present invention is directed to quantum dots that effectively kill multidrug-resistant bacteria that are simultaneously resistant to several types of antibiotics. Accordingly, the quantum dots are capable of effectively killing bacteria when used at a low concentration by optimizing the core bandgap thereof and also do not exhibit cytotoxicity, so they are useful as an agent for preventing or treating infectious diseases caused by multidrug-resistant bacteria.

Although specific embodiments of the present invention have been disclosed in detail above, it will be obvious to those skilled in the art that the description is merely of preferable exemplary embodiments, and is not to be construed as limiting the scope of the present invention. Therefore, the substantial scope of the present invention will be defined by the appended claims and equivalents thereof.

What is claimed is:

1. Inorganic nanoparticle quantum dots introduced with a hydrophilic ligand having activity of killing multidrug-resistant bacteria (MDR).

2. The inorganic nanoparticle quantum dots according to claim 1, wherein the inorganic nanoparticle quantum dots are quantum dots having an indium phosphide core/zinc selenide shell (InP/ZnSe) or an indium phosphide core/zinc sulfide shell (InP/ZnS).

3. The inorganic nanoparticle quantum dots according to claim 1, wherein the inorganic nanoparticle quantum dots have a core bandgap of 2 eV to 3 eV.

4. The inorganic nanoparticle quantum dots according to claim 1, wherein reactive oxygen species (ROS) generated by irradiating the inorganic nanoparticle quantum dots with light at a wavelength of 300 nm to 500 nm kills multidrug-resistant bacteria or inhibits growth thereof.

5. The inorganic nanoparticle quantum dots according to claim 1, wherein the hydrophilic ligand is selected from the group consisting of 3-mercaptopropionic acid (MPA), L-glutathione (GSH), mercaptoacetic acid, mercaptobutanoic acid, mercaptopentanoic acid, mercaptohexanoic acid, mercaptoheptanoic acid, mercaptooctanoic acid, mercaptononanoic acid, mercaptodecanoic acid, mercaptoundecanoic acid, mercaptododecanoic acid, and L-cysteine.

6. The inorganic nanoparticle quantum dots according to claim 1, wherein the multidrug-resistant bacteria is selected from the group consisting of Bacillus cereus, Staphylococcus aureus, Escherichia coli, Pseudomonas aeruginosa, Acinetobacter baumannii, Klebsiella pneumonia, Enterococcus faecium, Enterobacteriaceae, Helicobacter pylori, Campylobacter spp., Salmonellae, Neisseria gonorrhoeae, Streptococcus pneumoniae, Haemophilus influenzae, and Shigella spp.

7. A composition for killing multidrug-resistant bacteria or inhibiting growth thereof comprising the inorganic nanoparticle quantum dots according to claim 1.

8. A composition for preventing or treating an infectious disease caused by multidrug-resistant bacteria comprising the inorganic nanoparticle quantum dots according to claim 1.

9. The composition according to claim 8, wherein a concentration of the inorganic nanoparticle quantum dots is 50 nM to 200 nM.

10. The composition according to claim 8, wherein the infectious disease caused by multidrug-resistant bacteria is pneumonia, sepsis, urinary tract infection, food poisoning, impetigo, purulent disease, acute dermatitis, wound infection, bacteremia, endocarditis, or enteritis.

11. A method of killing multidrug-resistant bacteria using light, comprising:
(a) mixing the inorganic nanoparticle quantum dots according to claim 1 with multidrug-resistant bacteria in vitro; and
(b) radiating light onto the multidrug-resistant bacteria mixed with the quantum dots.

12. The method according to claim 11, wherein a concentration of the inorganic nanoparticle quantum dots in step (a) is 50 nM to 200 nM.

13. The method according to claim 11, wherein in step (b), light is radiated at a wavelength of 300 nm to 500 nm.
